(19) United States
(12) Patent Application Publication
(10) Pub. No.: US 2019/0175859 A1
(43) Pub. Date: Jun. 13, 2019

(54) SEAL-FORMING STRUCTURE FOR TREATING SLEEP DISORDERED BREATHING

(71) Applicant: Bespoke Medical Innovations Pty. Ltd., Baulkham Hills (AU)

(72) Inventors: Christopher DODDS, Baulkham Hills (AU); John HILTON, Baulkham Hills (AU); Philip KWOK, Baulkham Hills (AU)

(73) Assignee: Bespoke Medical Innovations Pty. Ltd., Baulkham Hills (AU)

(21) Appl. No.: 16/227,412

(22) Filed: Dec. 20, 2018

(30) Foreign Application Priority Data
Dec. 11, 2017 (AU) 2017904973

(51) Int. Cl. A61M 16/06

(52) U.S. Cl. CPC A61M 16/0616; A61M 16/0666; A61M 2207/00; A61M 2205/50; A61M 2016/0661

(57) ABSTRACT

A seal-forming structure (91) for treating sleep disordered breathing by delivering breathable gas to an entrance of a patient's airways during sleep at a pressure elevated above atmospheric pressure in a range of 4 to 20 cm H₂O, the seal-forming structure (91) comprising: a face-engaging surface (95) that is personalised to a patient's facial contour and to form a seal with the patient's face; and at least one comfort region (75, 80) configured to provide substantially even contact pressure between the face-engaging surface (95) against the patient's face when headgear tension is applied in use for maintaining the position of the seal-forming structure (91) on the patient's face; wherein the seal-forming structure (91) is non-deformable in response to headgear tension or pressurised air received within the seal-forming structure (91).

### FIELD OF THE INVENTION

The present invention relates to an improved seal-forming structure for treating sleep disordered breathing (SDB).

### BACKGROUND TO THE INVENTION

Sleep apnoea is a form of SDB and is commonly treated with equipment providing continuous positive airway pressure (CPAP), typically between 4 and 20 cm H₂O air pressure, to the nasal passage of the patient via a patient interface that forms a seal against the patient's face. The air pressure may be higher than 20 cm H₂O for bi-level positive airway pressure. CPAP acts as a pneumatic splint and may prevent upper airway occlusion by pushing the soft palate and tongue forward and away from the posterior oropharyngeal wall.

Changes in air pressure result in a change in the average contact pressure of a seal-forming structure positioned against the patient's face. A sealing coefficient is defined as the ratio of the change in average contact pressure to the change in air pressure minus one and is typically expressed as a percentage. The forces experienced by a seal-forming structure (for example, a rigid nasal adapter disclosed in WO2016/154676 which is expressly incorporated by reference herein in its entirety) are due to gravity; even pressures from ambient air and pressurized air from an interior space of the patient interface; and generally uneven contact pressure between seal-forming structure and the patient's skin. A single resultant 3D vector force can be calculated for each of these items. The vector sum and the moment sum of these vector forces are both zero. To approximate and simplify, gravity is insignificant and pressure values are taken relative to ambient air pressure. This leaves three key 3D vector forces: the resulting single force on the rigid nasal adapter due to contact with the mask, the resulting single force due to contact with the pressurized air and the resulting single force due to contact with the patient's skin.

The mask force and the pressurized air force combine to push the rigid nasal adapter onto the patient's face and the skin force being equal and opposite to oppose this push. The 3D axis of the opposing force from the patient's skin determines the axis from which the pressurized air and skin contact cross sectional areas are calculated.

For a patient interface where the seal-forming structure is a flexible mask cushion, the combined push vector can be calculated as the largest pressurized air cross section multiplied by the air pressure. Prior masks with a flexible and deformable mask cushion have used increased air pressure to produce a stronger push onto a stiffer peripheral wall portion or rim of the flexible mask cushion to provide a stronger sealing force with higher air pressures. One drawback is that such a flexible mask cushion is bulky, is not visually aesthetic and obstructs a relatively large portion of the patient's face. Alternate prior masks have large skin contact areas and/or require a high level of headgear tension for their mask straps and therefore very uncomfortable for patients because CPAP therapy is typically required for prolonged duration, at least 5 hours each night.

Prior masks with a flexible and deformable mask cushion require headgear straps to be highly tensioned until a perimeter seal is achieved by the mask cushion. Areas of interference are further compressed. These prior masks attempt to make these areas of interference flex more to accommodate population variation of facial anthropometric differences. A rigid seal-forming structure results in minimal or nominal concentrated facial compression because the seal-forming structure is personalised or customised for an individual patient without high or low gaps between the seal-forming structure and the patient's face. Therefore headgear tension used for a rigid seal-forming structure can be lower relative to prior masks with deformable or soft mask cushions, resulting in high levels of comfort without facial marking (red marks) for the patient.

### SUMMARY OF THE INVENTION

The inventive concept arises from a recognition that a rigid seal-forming structure can be comfortable for a patient.

The present invention, in one aspect, comprises a seal-forming structure for treating sleep disordered breathing by delivering breathable gas to an entrance of a patient's airways during sleep at a pressure elevated above atmospheric pressure in a range of 4 to 20 cm H₂O. The seal-forming structure comprises a face-engaging surface that is personalised to a patient's facial contour and to form a seal with the patient's face. The seal-forming structure also comprises at least one comfort region configured to provide substantially even contact pressure between the face-engaging surface against the patient's face when headgear tension is applied in use for maintaining the position of the seal-forming structure on the patient's face. The seal-forming structure is non-deformable in response to headgear tension or pressurised air received within the seal-forming structure.

The at least one comfort region may be determined based on its location in use proximal to a predetermined facial landmark.

The at least one comfort region may be determined based on predicted skin response or predicted tissue response when headgear tension is applied for maintaining the position of the seal-forming structure on the patient's face.

The at least one comfort region may be determined based on a predicted deformed condition of a patient's facial substructure when headgear tension is applied for maintaining the position of the seal-forming structure on the patient's face.

The at least one comfort region may be determined based on the presence or absence of facial hair.

The seal-forming structure may be part of an adapter for a patient interface.

The seal-forming structure may be a part of a nasal patient interface.

The at least one comfort region may be a first region that in use is proximal to a base region of the patient's septum adjacent to the patient's upper lip.

The at least one comfort region may be a second region that in use is proximal to a nasal bridge region of the patient's nose.

The at least one comfort region may be a third region that in use is proximal to a nose tip region of the patient's nose.

The at least one comfort region may have a depth from 0.1 mm to 5 mm.

The face-engaging surface is preferably personalised via a digitising process.

The present invention, in another aspect, comprises a method for manufacturing a seal-forming structure for treating sleep disordered breathing at a pressure elevated above atmospheric pressure in a range of at least 4 cm H₂O. The method comprises personalising a face-engaging surface to a patient's facial contour and to form a seal with the patient's face. The method also comprises forming at least one comfort region configured to provide substantially even contact pressure between the face-engaging surface against the patient's face when headgear tension is applied in use for maintaining the position of the seal-forming structure on the patient's face. The seal-forming structure is non-deformable in response to headgear tension or pressurised air received within the seal-forming structure.

The method may further comprise initial steps of: capturing images of the patient's face; and generating a three-dimensional (3D) model using the captured images.

The seal-forming structure may be manufactured using an additive manufacturing process.

Rigid in the context of the present invention means non-deformable in response to headgear tension or pressurised air received within the seal-forming structure. In a preferred form, rigidity of seal-forming structure for a patient interface for treating sleep apnea is measured in terms of deformation the seal-forming structure under a typical treatment condition that is simulated. The patient interface is rest against a simulated patient's face with loosened headgear straps and no air flow or treatment pressure. The distance is measured between the plane of the alar nasal sulci and the distal end of the skin contacting surface of the seal-forming structure of the patient interface to the nearest 0.5 mm. The patient interface is next connected to a flow generator and a treatment pressure of 20 cm H₂O air pressure is applied. The headgear straps are tightened such that there are no detectable leaks between the patient interface and the patient's face. Again, the distance is measured between the plane of the alar nasal sulci and the distal end of the skin contacting surface of the seal-forming structure of the patient interface to the nearest 0.5 mm

Existing masks such as the ResMed AirFit N20 nasal mask and Fisher & Paykel Eson nasal mask are compared to the present invention.

| | Distance from plane of alar nasal | | |
|---|---|---|---|
| | sulci to distal end of skin contacting | | |
| | mask component (mm) | | |
| | Loose headgear | Tightened | Average |
| Mask | straps | headgear straps | compression |
| ResMed AirFit | 55 mm average | 49.7 mm average | 5.3 mm |
| N20 | | | |
| Fisher & Paykel | 62.2 mm average | 55.5 mm average | 6.7 mm |
| Eson | | | |
| Present Invention | 39.8 mm average | 39.5 mm average | 0.3 mm |

Referring to the comparative table above, the present invention has a significantly lower compression of 0.3 mm on average compared to over 5 mm for existing patient interfaces comprising a deformable silicone cushion as a seal-forming structure. Therefore, rigidity of the seal-forming structure in the context of the present invention means having an average compression of less than 1 mm.

Other advantages and features according to the invention will be apparent to those of ordinary skill upon reading this application.

### BRIEF DESCRIPTION OF THE DRAWINGS

Embodiments of the invention will be described with respect to the figures, in which like reference numbers denote like elements and in which:

FIG. 1 is a screenshot of a face mesh generated from a 3D scan of a patient's face illustrating locations of the patient's face where comfort regions are added in accordance with an embodiment of the present invention;

FIG. 2 is a screenshot showing the sagittal anatomical plane and transverse anatomical plane;

FIG. 3 is a screenshot of a patient's face in a plane parallel to the sagittal anatomical plane before stretching for additional nose tip comfort;

FIG. 4 is a screenshot of a patient's face in a plane parallel to the transverse anatomical plane before stretching for additional nose tip comfort;

FIG. 5 is a screenshot of a patient's face in a plane parallel to the sagittal anatomical plane after stretching for additional nose tip comfort;

FIG. 6 is a screenshot of a patient's face in a plane parallel to the transverse anatomical plane after stretching for additional nose tip comfort;

FIG. 7 is a screenshot of a face mesh of a patient depicting a smoothing process to smooth the patient's alar nasal sulcus;

FIG. 8 is a screenshot depicting an offsetting process for the patient's nasal septum;

FIG. 9 is a front perspective view of a nasal adapter with a seal-forming structure in accordance with an embodiment of the present invention;

FIG. 10 is a rear perspective view of a nasal adapter with a seal-forming structure in accordance with an embodiment of the present invention;

FIG. 11 is a another front perspective view of a nasal adapter with a seal-forming structure in accordance with an embodiment of the present invention;

FIG. 12 is a rear view of a nasal adapter with a seal-forming structure in accordance with an embodiment of the present invention;

FIG. 13 is a bottom view of a nasal adapter with a seal-forming structure in accordance with an embodiment of the present invention;

FIG. 14 is a front view of a nasal adapter with a seal-forming structure in accordance with an embodiment of the present invention;

FIG. 15 is a side view of a nasal adapter with a seal-forming structure in accordance with an embodiment of the present invention;

FIG. 16 is a top view of a nasal adapter with a seal-forming structure in accordance with an embodiment of the present invention; and

FIG. 17 is a perspective view of a patient interface system comprising the nasal adapter of FIG. 9.

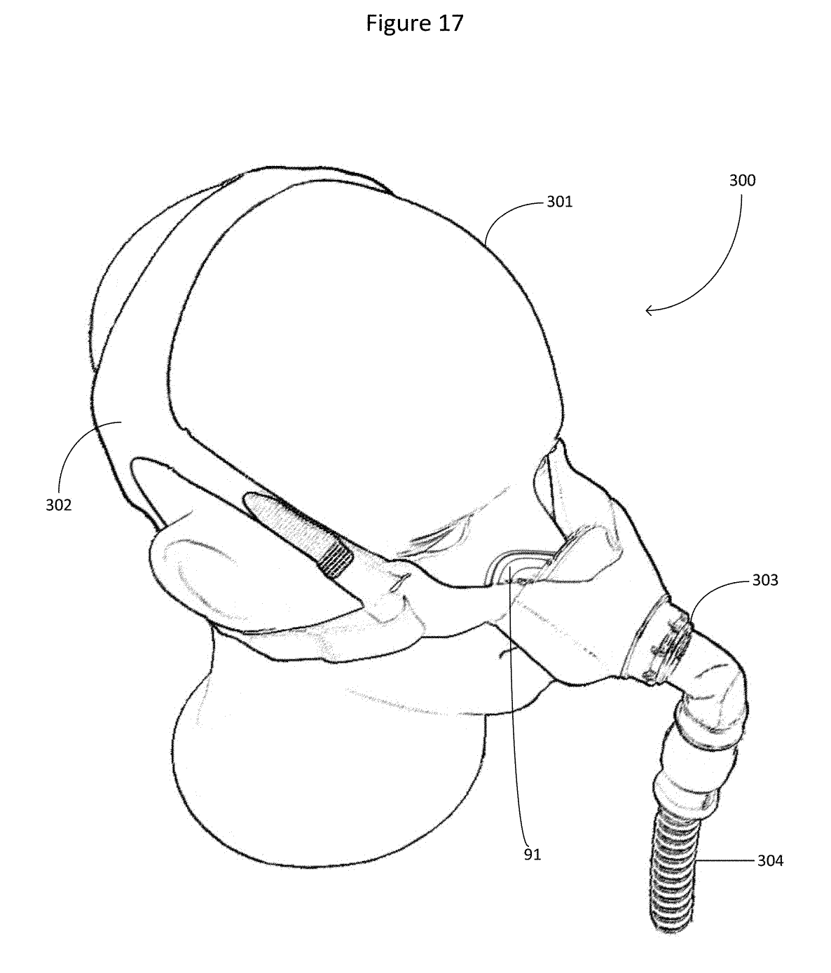

### DETAILED DESCRIPTION OF THE INVENTION

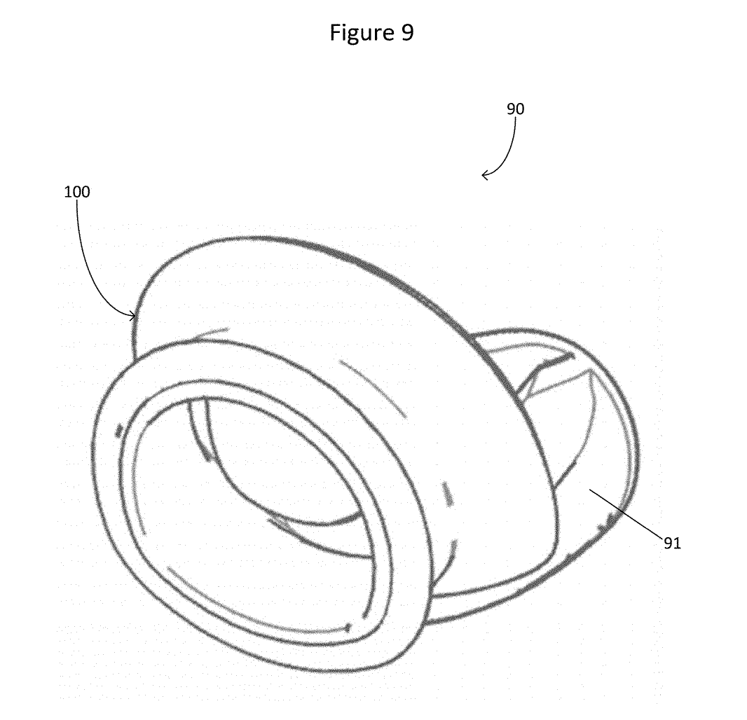

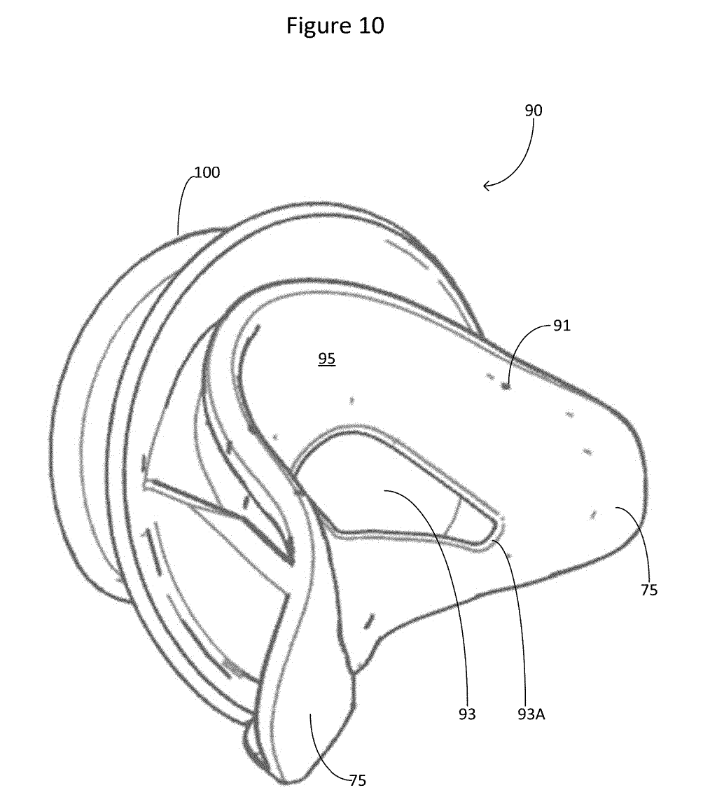

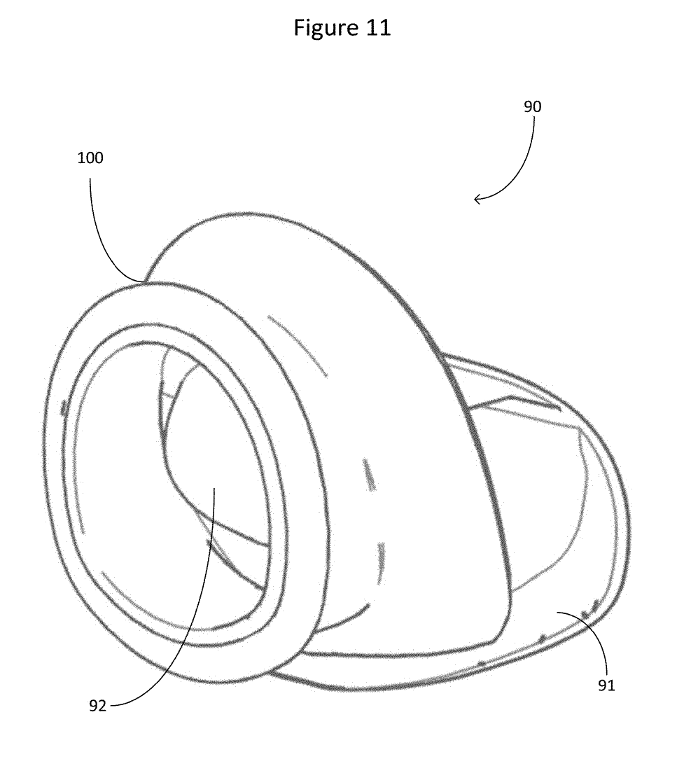

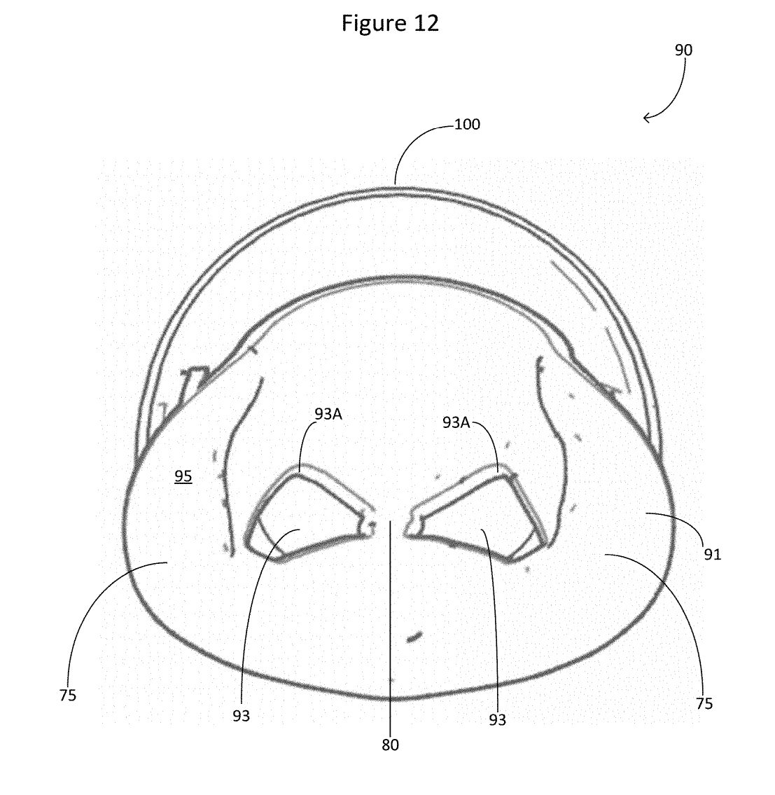

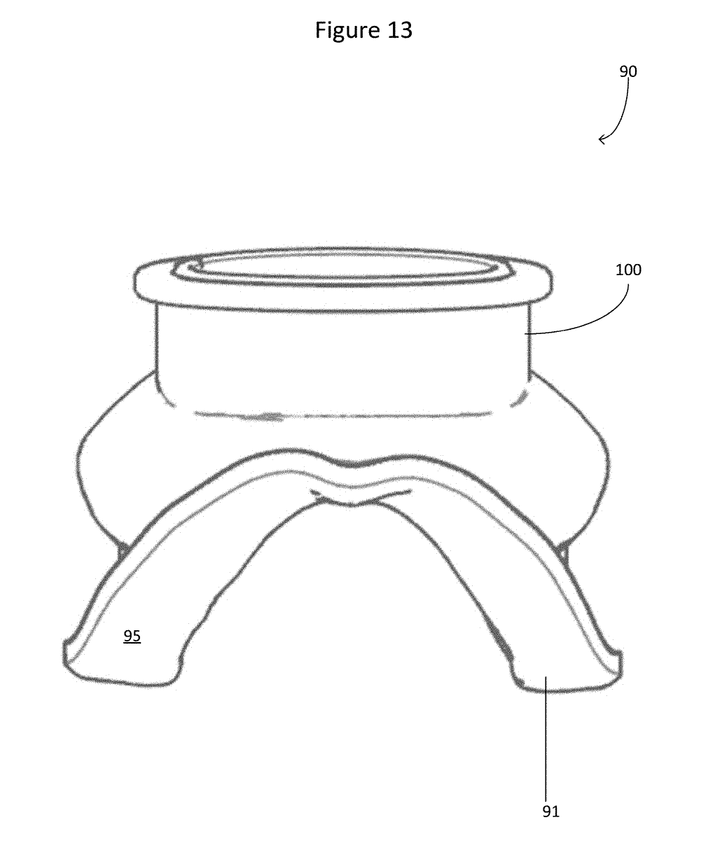

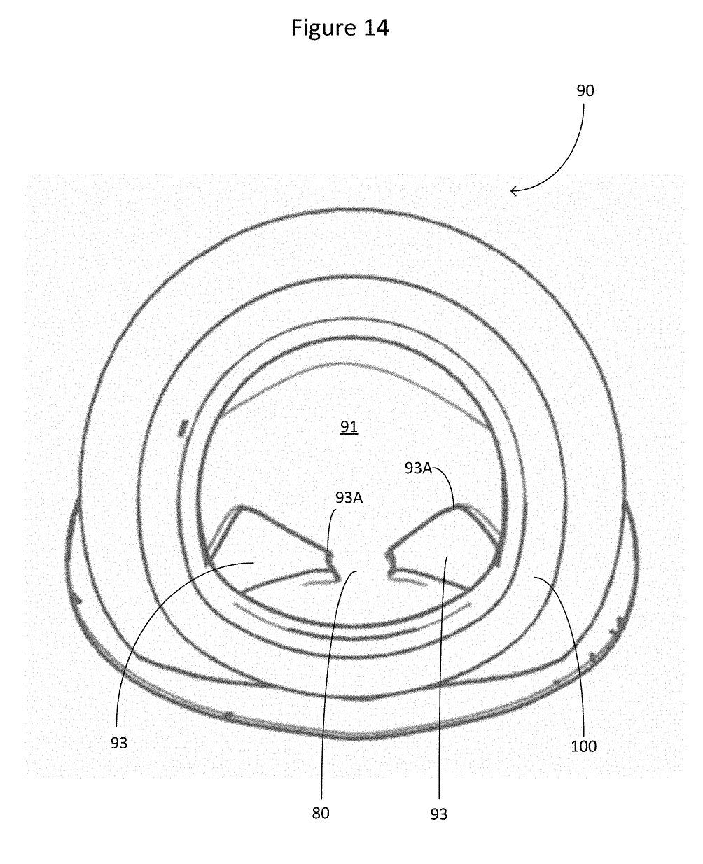

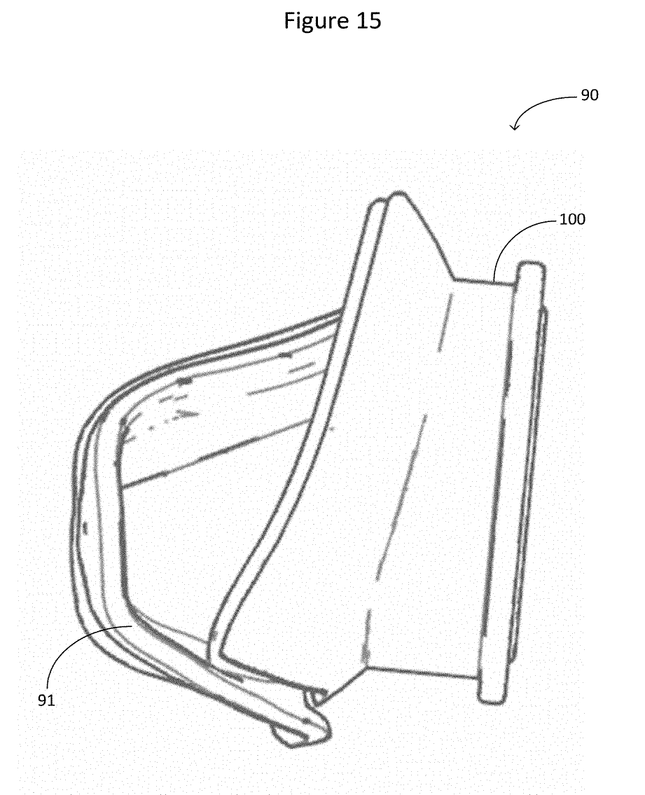

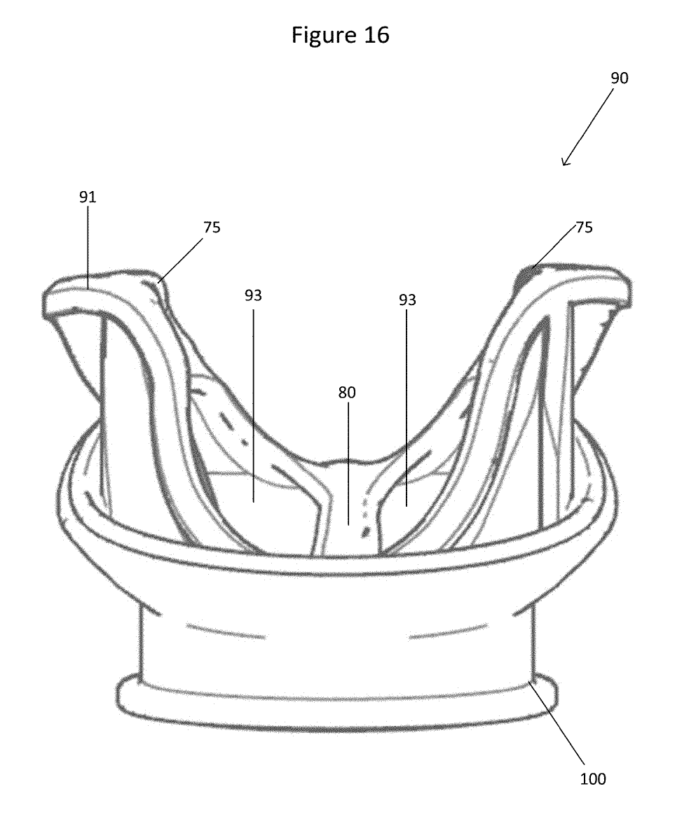

A preferred seal-forming structure 91 for treating sleep disordered breathing by delivering breathable gas to an entrance of a patient's airways during sleep at a pressure elevated above atmospheric pressure in a range of 4 to 20 cm H₂O according to the present invention is illustrated in FIGS. 9 to 16. A nasal adapter with the seal-forming structure 91 is shown generally at reference numeral 90. The seal-forming structure 91 is part of a patient interface or a nasal adapter for a patient interface 300 and in use is arranged to surround an entrance to the airways of the patient 301 so as to facilitate the supply of air at positive pressure to the airways. The seal-forming structure 91 extends in use about the entire perimeter of a plenum chamber 92. The plenum chamber 92 is a portion of the patient interface 300 having walls enclosing a volume of space which has air therein pressurised above atmospheric pressure in use. The plenum chamber 92 may be flexible, semi-rigid or not readily deformable to finger pressure (or from pressurised air within the plenum chamber 92). The patient interface 300 also comprises headgear 302 to retain the patient interface 300 against the patient's face during therapy. The headgear 302 functions as a positioning and stabilising structure for use on a patient's head. The headgear 302 may comprise ties (e.g. formed of soft flexible elastic material) and stiffeners (to limit flexibility in certain directions or elongation in certain directions). The patient interface 300 has or is operatively connectable to an air conduit 304 to deliver air at positive pressure from a positive airway pressure (PAP) device (not shown). The patient interface 300 also comprises at least one vent 303 to enable an intentional flow of air from an interior of the patient interface 300 to ambient air for the purpose of allowing washout of exhaled gases by the patient 301.

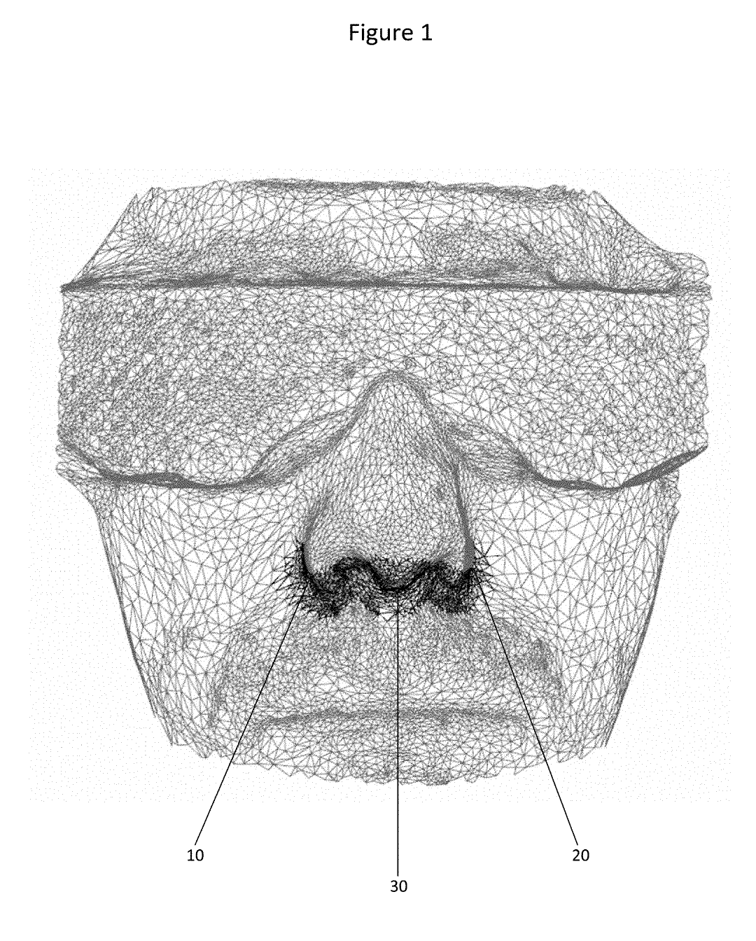

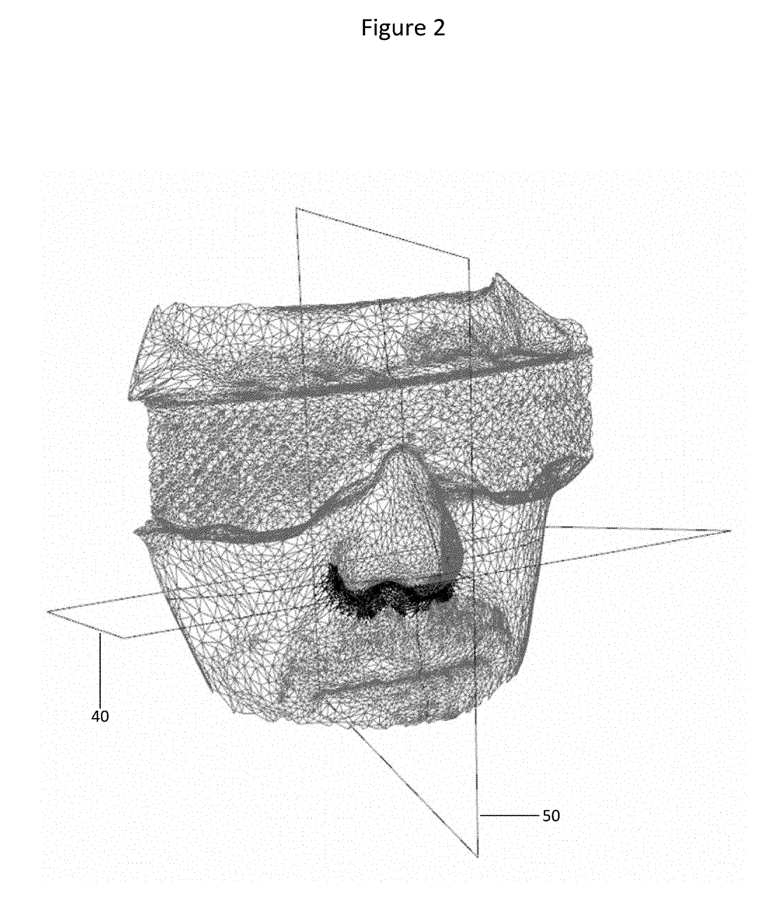

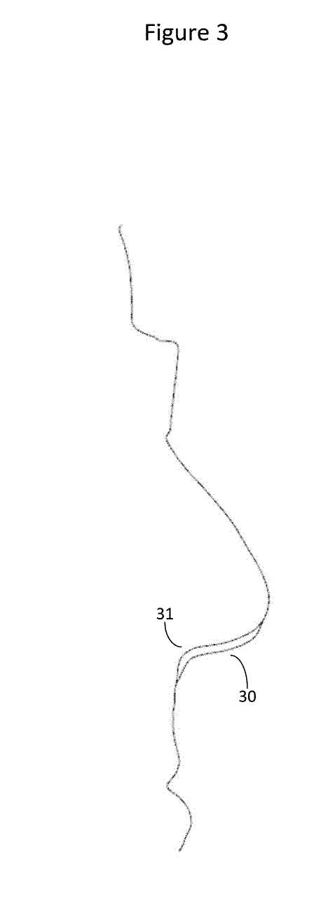

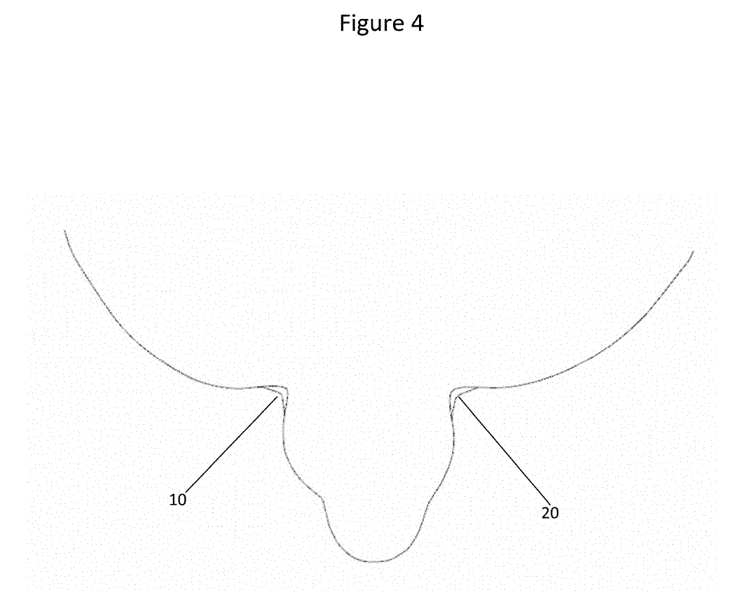

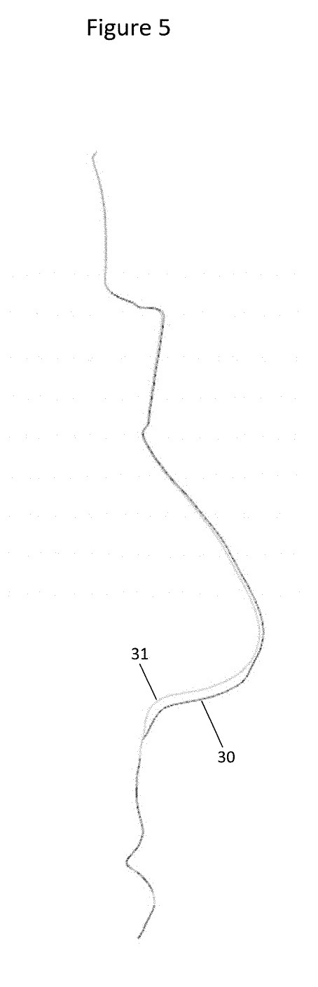

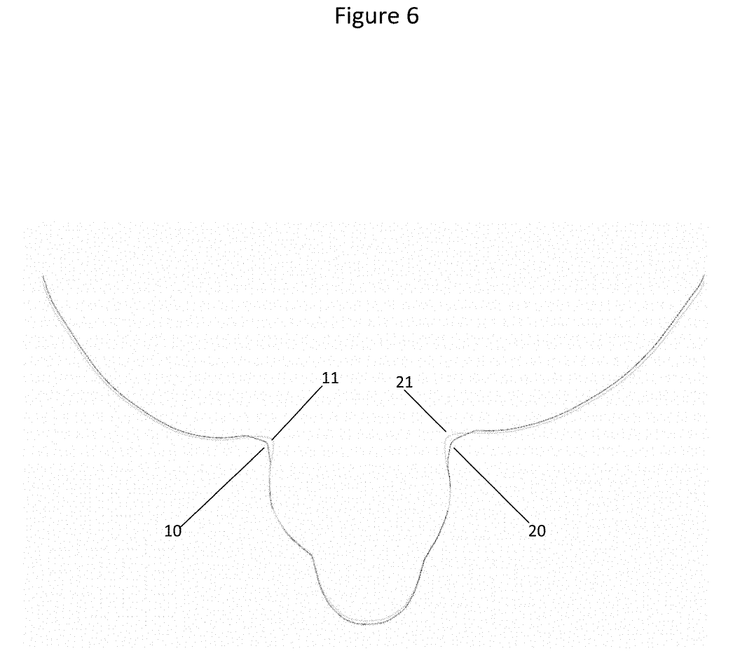

Referring to FIGS. 1 to 6, the original 3D scan of the patient's face is indicated in grey colour 11, 21, 31, and the modified patient's face with comfort regions is indicated in black colour 10, 20, 30. The comfort regions are offsets (positive or negative) at particular areas or points of the seal-forming structure 91 that are offset from the identical original 3D scan of the patient's face to improve the comfort of the seal-forming structure 91 when worn by the patient 301 undergoing therapy over extended duration. The modified 3D model of the patient's face is used to generate a 3D model of a rigid seal-forming structure 91 which can be manufactured using an additive manufacturing process such as 3D printing. The rigid seal-forming structure 91 may be made from acrylonitrile butadiene styrene (ABS) plastic, polycarbonate plastic, polyurethane, poly lactic acid (PLA) plastic or polyethylene plastic.

The geometry and location of the comfort regions 75, 80 are selected and determined to provide substantially even contact pressure between a face-engaging surface of the seal-forming structure 91 against the patient's face when headgear tension is applied in use for maintaining the position of the seal-forming structure 91 on the patient's face. Uneven contact pressure or excessive contract pressure can cause concentration of forces against the patient's face that can lead to sores and red marks after prolonged wearing of the patient interface 300. Headgear tension may be adjustable depending on the tightening of the headgear 302 from length adjustment of headgear straps or may be auto-adjusting depending on the elasticity and stretch profile of the headgear strap material. The comfort regions 75, 80 also improve patient comfort by enabling a minimal contact pressure between the seal-forming structure 91 and the patient's face when headgear tension is applied to retain the patient interface against the patient's face in use.

An acceptable and comfortable contact pressure can be subjectively determined from patient feedback or objectively determined, for example, by estimating critical closing pressure (CCP) of vessels supplying the capillary loops of a patient's face. The epidermis at the patient's nose is about 75 microns thick and almost devoid of rete pegs. There are about 55 to 148 capillary mounds per square millimetre of epidermis. The vascular loops occur under the epidermis at regular intervals. At the area of the philtrum including the nasolabial folds the epidermis is 35 to 100 microns thick. The number of capillary loops per square millimetre of epidermis is about 92 to 222. The papillary body of the face has a rich capillary bed. An objective determination of comfortable contact pressure for a specific patient is visually ascertainable based on the change in the colour of the patient's skin before a load is applied compared to the colour of the patient's skin when a load is applied (for example, a patient interface 300 held by headgear tension and providing therapeutic pressure) for a predetermined duration (for example, therapy duration of at least 3.7 hours). Presence or absence of blood flow in the capillary loops can be detected by direct microscopic observation of the movement of cells within the loops, for example, using a high resolution camera with a zoom lens. A simulation of a hypothetical load can be performed on a patient, for example, at the same time the patient's face is 3D scanned, and measurements are taken and analysed. If the contact pressure is high enough to cause discomfort to the patient and lead to red marks and sores after wearing the patient interface 300 for extended duration, then this is unacceptable. The structure and surface profile of the seal-forming structure 91 is configured to provide a substantially even contact pressure through the provision of comfort regions in the seal-forming structure 91 that do not result in vessels supplying the capillary loops of a patient's face to fall below the estimated CCP.

A face mesh or 3D model (see FIG. 1) is generated from a plurality of digital photographs capturing the patient's face. This is a digitising process. An exemplary portable photogrammetry studio suitable for taking photos of the patient's face is disclosed in WO2017/187661 which is expressly incorporated by reference herein in its entirety. In one embodiment, 20 photos of the patient are stitched together to create the 3D model using photogrammetry software. The photogrammetry software is executed by a computer comprising a central processing unit (CPU) or graphics processing unit (GPU) specialised for display functions capable of handling thousands of threads simultaneously. The 3D model is textured with wireframe. The face mesh is trimmed to remove excess parts or features such as black braces at the patient's forehead and chin. The trimmed face mesh is exported for further processing by the computer.

The exported face mesh is imported by a 3D sculpting-based computer aided design (CAD) software for modification to generate comfort regions in order to provide a substantially even contact pressure between the customised seal-forming structure against the patient's face in use. This may also improve the comfort of the customised seal-forming structure 91 in use. The face mesh is scaled and oriented correctly based on landmarks present in photos/3D model using the photogrammetry software. For example, there are three circular landmarks provided on glasses worn by the patient at the time the photos are taken. The landmarks have a known distance apart from each other and are at known angles from each other. The landmarks are detectable in the 3D model by the CAD software.

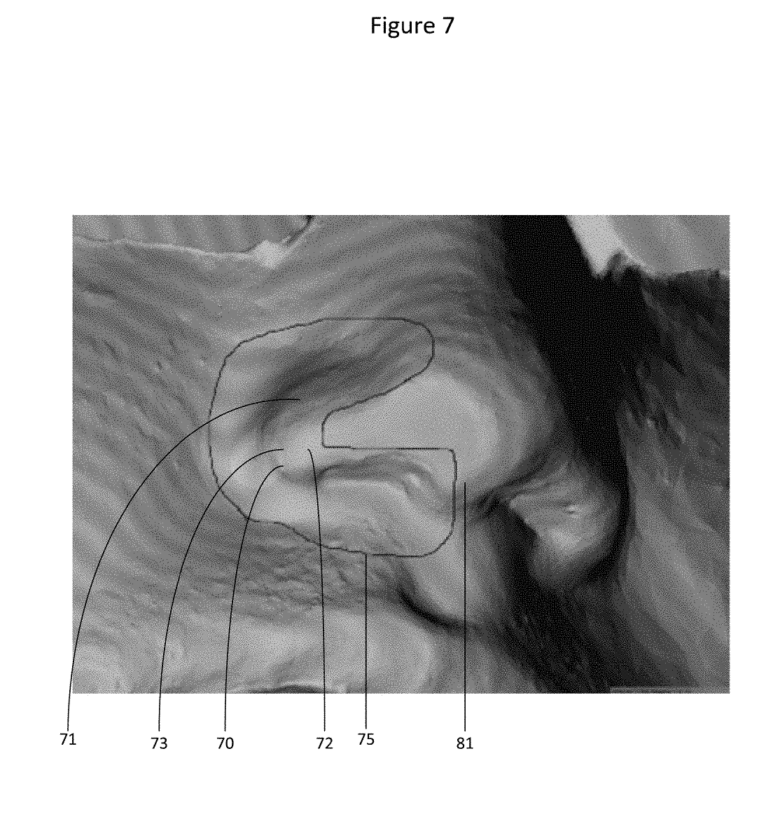

Referring to FIG. 7, both of the patient's alar nasal sulcus 70 are identified in the face mesh. A smoothing process is executed for each identified alar nasal sulcus 70 and smooths the region around the alar nasal sulcus 70, alar groove 71, alar lobule 72 and ala nasi. The smoothing process creates a gradual arc or curved comfort region 75 from about 0.1 mm to 5 mm indicated by the solid outline illustrated in FIG. 7. The rate of smoothing and the exact distance for the comfort region 75 can be calibrated depending on the patient's comfort preference level.

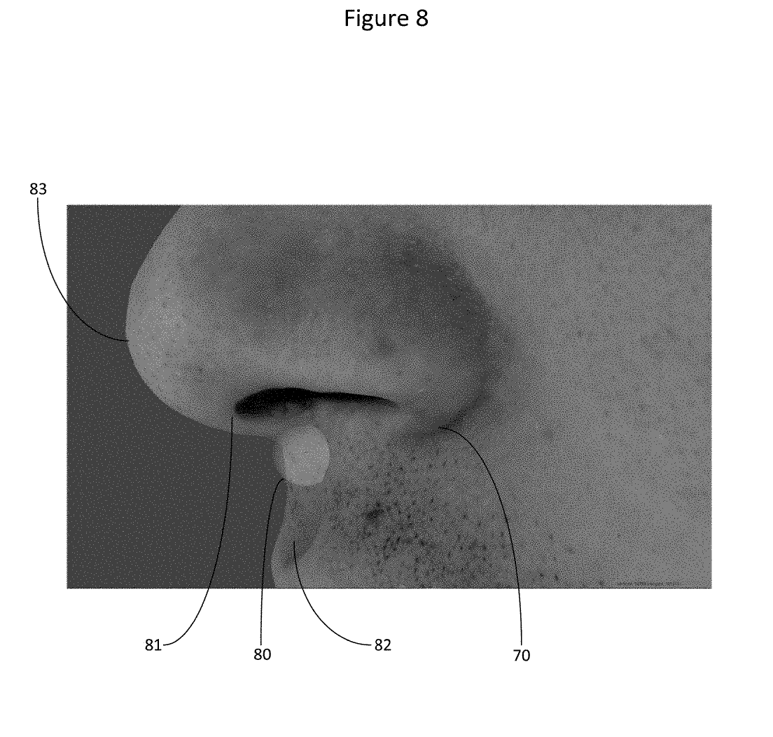

Referring to FIG. 8, the patient's nasal septum 81 is identified in the face mesh. An offsetting process is executed which creates a gradual comfort region 80 from about 0.1 mm to 5 mm below the nasal septum 81 proximal to the middle of the patient's philtrum 82. The exact distance for the comfort region 80 can be calibrated depending on the patient's comfort preference level. In one embodiment, the offsetting process is executed for the patient's nose tip (apex of nose) 83 and nasal bridge (dorsum nasi) in a similar fashion.

In one embodiment, the identification and classification of facial features in the face mesh is performed by a mesh slicing algorithm. The orientation of the face mesh is estimated using the position of known points on eyewear worn by the patient during the face capture process performed, for example, by a portable photogrammetry studio. There may be 3 known points on the eyewear to determine real-world scale and angular rotation. The face mesh is then sliced along a number of different predetermined planes to obtain cross-sections. Key features of the patient's face are detected from the curvature of these cross-sections. These key features are used to construct the skin contacting surface of the seal-forming structure 91 and to position the connections of the seal-forming structure 91 to the non-customised/non-personalised components of the patient interface 300, for example, a mask frame/mask chassis or lock ring.

In another embodiment, the identification and classification of facial features in the face mesh can be performed by a machine learning point cloud classification algorithm. The machine learning point cloud classification algorithm is trained using training data to build a facial features classification model and this may be supervised or semi-supervised. The algorithm is trained based on geometry and pixel values to understand object classes. 3D points in the face mesh are automatically classified, for example, as being the left alar nasal sulcus, nasal septum, philtrum, etc.

It is envisaged that other locations and areas of the patient's face may benefit from a comfort region to provide substantially even contact pressure between a face-engaging surface 95 of the seal-forming structure 91 against the patient's face and improve patient comfort. Another area includes towards the patient's face to create a region of interference along the outer/peripheral rim of the seal-forming structure 91. Another area includes away from the patient's face, above and over the top of the nare apertures 93 of the seal-forming structure 91. Another area includes along the peripheral edges 93A of the nare apertures 93 to gently and gradually curve away. Other areas that may have a comfort region which are dependent on individual patient facial characteristics include bony areas of the patient's face, areas of the patient's face with reduced soft tissue thickness or areas where skin is thinner. Providing light (low force) and even contact pressure at these sensitive facial locations can ameliorate or avoid pressure sores and red marks caused by irritation from prolonged wearing of the patient interface from a concentration of higher levels of contact pressure at localised positions. An area where there is considered a high level of soft tissue thickness is the patient's cheeks (slightly below the cheekbones). Contact pressure at this area can be absorbed more comfortably by the patient because there can be more compression and displacement of the soft tissue in the cheeks.

A predicted deformed condition of a patient's facial substructure when headgear tension is applied for maintaining the position of the seal-forming structure on the patient's face can be considered when determining the location for a comfort region. A pressure sensor or tactile force sensor can be used to generate a pressure map of the patient's face and determine the deformed condition of a patient's facial substructure. The elasticity of the patient's face at different locations indicates areas where there is higher sensitivity when a load or force is exerted from headgear tension urging the patient interface and seal-forming structure 91 against the patient's face. These high sensitivity zones are more likely to benefit from having a comfort region located at a corresponding position in the seal-forming structure. The comfort region(s) enable the seal-forming structure 91 from digging into the patient's face and concentrating contact pressure at these high sensitivity zones.

The patient may have facial hair such as a beard or moustache. Facial hair captured on the photos is detectable in the 3D model and the extent of facial hair can be quantified. The geometry and depth of a comfort region can be determined based on the presence or absence of facial hair. A further modification to the offset (positive or negative) to generate a comfort region for facial hair can be provided if facial hair e.g. a moustache, is detected that will coincide with the seal path of the customised seal-forming structure 91. The facial hair can be detected in the 3D model, and a new facial surface that is smoothed can estimate the patient's facial surface with the absence of facial hair. The comfort region for facial hair is generated by applying an offset (positive or negative) from the estimated patient's facial surface in the areas where facial hair is detected. It has been difficult for prior masks with flexible cushions to seal adequately when a patient has facial hair because small air gaps can occur along with the seal path due to the presence of hair and lead to significant loss of therapy pressure, even where a tighter tension for the headgear is applied. The combination of a patient interface with a customised rigid seal-forming structure 91 provided with intelligently located and shaped comfort regions for facial hair ameliorates this difficulty and can provide an adequate seal for patients with facial hair that substantially maintains therapy pressure with nominal leak. Although there may be no actual seal, the comfort regions for facial hair does not degrade the level of pressure below a therapeutic level and therefore therapeutic pressure can be maintained for patients with facial hair. In one example, the facial hair is squashed down against the patient's skin when the seal-forming structure is 91 is worn. Any nominal leak is only to the extent that it does not arouse or annoy the patient or the patient's bed partner.

The patient's nostrils are identified in the face mesh. The shape of the nostrils are identified and marked as nostril meshes to be removed. A curve generation process is applied to the outline of the nostril meshes such that the peripheral edge of the nostril meshes has increased curvature. Creating curved surfaces rather than leaving sharp angular surfaces to remain improves comfort by preventing sore spots from occurring. The curved nostril meshes are removed from the face mesh. After removal of the nostril meshes, two nare apertures 93 are provided in the seal-forming structure 91 that correspond to the location of the patient's nostrils and align with the orientation of the patient's nostrils. A 3DM file of a generic seal-forming structure that is not personalised yet is used as an initial canvas. There is a model of the subcomponent of the seal-forming structure 91 that interfaces with the mask 300. This model needs to align with the now modified face mesh. In one example, the model is positioned so that it centred just below the tip of the patient's nose, the angle and scale of the interface is constant, and the interface is moved to sit as close to the modified face mesh of the patient's face as it can. After the model is located in this position, the model is combined with the face mesh using a sequence of Boolean operations. Since the cross-sectional area of the two nare apertures 93 are substantially the same as the patient's nares or slightly larger, there is less likelihood of a side effect known as air jetting where air enters the nose in a channelled manner and impinges on sensitive nasal mucosa. Also, the patient's septum 81 has some comfort relief because it is not in the direct flow path of the incoming air via the plenum chamber 92 because of the existence of material (with comfort region 80) separating the two nare apertures 93.

After the modified face mesh with the two curved nostril meshes removed is completed, a 3D mesh of a rigid seal forming structure is generated that fits to the modified face mesh. The 3D mesh of the seal forming structure is aligned and best fit against the modified face mesh and the patient's nostrils in the mesh are completely enclosed by the seal forming structure mesh. A trim process is applied on the 3D mesh of the seal forming structure to remove any excess material and to make the resulting seal forming structure as minimal as possible such that is it visually unobtrusive and also light-weight. If the seal forming structure 91 is intended to be used as a nasal adapter 90, a 3D mesh of a lock ring 100 is generated that joins with the 3D mesh of the seal forming structure. The lock ring 100 enables connection of the seal-forming structure 91 with an existing patient interface. A check process is performed to ensure that that the final 3D mesh of the seal forming structure (with or without the lock ring) is a single solid piece with no naked edges. If the check process is passed, the final 3D mesh of the seal forming structure is exported (e.g. as an STL file) to a 3D printer for manufacturing.

In one embodiment, photogrammetry software is configured to execute computer executable instructions to construct a 3D model of the patient's face from photographs taken of the patient's face by a portable photogrammetry studio. The portable photogrammetry studio uploads photographs, for example, at least 20 photographs of a patient's face via the Internet to cloud storage or a server. The photographs may be in JPG format. The photogrammetry software automatically detects the newly uploaded photographs on a network folder it monitors and executes a conversion process to construct a 3D model of the patient's face based on the photographs. In another embodiment, the 3D models of multiple patients can be constructed in a daily batch process instead of on a demand basis. If the photographs are deficient in that a 3D model cannot be constructed, for example, due to poor lighting or the presence of too much motion blur, the portable photogrammetry studio can be prompted to initiate another set of photographs be taken. If a 3D model can be successfully constructed, the 3D model is saved as an OBJ file. Next, an interpreted program automates the execution of tasks to process the 3D model (e.g. the OBJ file). Tasks include scaling the face mesh in the 3D model as described earlier by identification of known landmarks detected in the 3D model (e.g. 3 circular landmarks at known distance and known angles from each other on glasses worn by the patient when the photographs are taken). The outline of the skin contacting area is defined. Another task performs the offsetting and generation of comfort regions in areas of the face mesh where clearance/interference is required to provide substantially even contact pressure between the seal-forming structure 91 against the patient's face. The nostril meshes are removed corresponding to nare apertures 93 from the face mesh. Another task imports, positions and fits a 3D model (e.g. a 3DM file) of the patient interface or seal-forming structure relative to the modified face mesh. If the seal forming structure 91 is used as a nasal adapter 90, the seal-forming structure mesh is combined with a lock ring mesh to generate a 3D model of the nasal adapter 90. Lastly, the interpreted program outputs the 3D model with the comfort regions 75, 80 of the seal forming structure 91 or nasal adapter 90 with the seal forming structure 91 (as a STL file), to be manufactured by a 3D printer.

A further improved seal-forming structure maps the patient's face to identify which regions can compressed further. An air puff tonometer is used to create a map of the mechanical properties of tissues of the patient's face.

Although the comfort regions 75, 80 have been described as being determined and formed in a subtractive manner, it is envisaged that the process can be achieved by initially subtracting a predetermined amount from the original face mesh and later adding seal-forming structure regions to the face mesh except for areas that require an offset (positive or negative) to enable substantially even contact pressure between the rigid seal-forming structure 91 against the patient's face.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements.

It is therefore to be understood that numerous modifications may be made to the illustrative examples and that other arrangements may be devised without departing from the spirit and scope of the technology.

1. A seal-forming structure for treating sleep disordered breathing by delivering breathable gas to an entrance of a patient's airways during sleep at a pressure elevated above atmospheric pressure in a range of 4 to 20 cm H₂O, the seal-forming structure comprising:
a face-engaging surface that is personalised to a patient's facial contour and to form a seal with the patient's face; and
at least one comfort region configured to provide substantially even contact pressure between the face-engaging surface against the patient's face when headgear tension is applied in use for maintaining the position of the seal-forming structure on the patient's face;
wherein the seal-forming structure is non-deformable in response to headgear tension or pressurised air received within the seal-forming structure.

2. The seal-forming structure according to claim 1, wherein the at least one comfort region is determined based on its location in use proximal to a predetermined facial landmark.

3. The seal-forming structure according to claim 1, wherein the at least one comfort region is determined based on predicted skin response or predicted tissue response when headgear tension is applied for maintaining the position of the seal-forming structure on the patient's face.

4. The seal-forming structure according to claim 1, wherein the at least one comfort region is determined based on a predicted deformed condition of a patient's facial substructure when headgear tension is applied for maintaining the position of the seal-forming structure on the patient's face.

5. The seal-forming structure according to claim 1, wherein the at least one comfort region is determined based on the presence or absence of facial hair.

6. The seal-forming structure according to claim 1, wherein the seal-forming structure is part of an adapter for a patient interface.

7. The seal-forming structure according to claim 1, wherein the seal-forming structure is a part of a nasal patient interface.

8. The seal-forming structure according to claim 2, wherein the at least one comfort region is a first region that in use is proximal to a base region of the patient's septum adjacent to the patient's upper lip.

9. The seal-forming structure according to claim 2, wherein the at least one comfort region is a second region that in use is proximal to a nasal bridge region of the patient's nose.

10. The seal-forming structure according to claim 2, wherein the at least one comfort region is a third region that in use is proximal to a nose tip region of the patient's nose.

11. The seal-forming structure according to any one of claims 8 to 10, wherein the at least one comfort region has a depth from 0.1 mm to 5 mm.

12. The seal-forming structure according to claim 1, wherein the face-engaging surface is personalised via a digitising process.

13. A method for manufacturing a seal-forming structure for treating sleep disordered breathing at a pressure elevated above atmospheric pressure in a range of at least 4 cm H₂O, the method comprising:
personalising a face-engaging surface to a patient's facial contour and to form a seal with the patient's face; and
forming at least one comfort region configured to provide substantially even contact pressure between the face-engaging surface against the patient's face when headgear tension is applied in use for maintaining the position of the seal-forming structure on the patient's face;
wherein the seal-forming structure is non-deformable in response to headgear tension or pressurised air received within the seal-forming structure.

14. The method according to claim 13, further comprising initial steps of:
capturing images of the patient's face; and
generating a three-dimensional (3D) model using the captured images.

15. The method according to claim 13, wherein the seal-forming structure is manufactured using an additive manufacturing process.
